SEQUENCE LISTING (12 sequences)

SEQ ID NO: 1 - DNA, 26 bp, Artificial Sequence
  Primer
cagtggacatgtccggtgggacggtc

SEQ ID NO: 2 - DNA, 27 bp, Artificial Sequence
  Primer
ttcttggcaacggcaacaaaccacaac

SEQ ID NO: 3 - DNA, 21 bp, Artificial Sequence
  Primer
ccacaccgtttagcggaatgc

SEQ ID NO: 4 - DNA, 25 bp, Artificial Sequence
  Primer
cgggactctaagatgaagttatggg

SEQ ID NO: 5 - DNA, 22 bp, Artificial Sequence
  Primer
gttttggccagtggtcgtgcag

SEQ ID NO: 6 - DNA, 22 bp, Artificial Sequence
  Primer
ccgcttgctcctgtgagtcctg

SEQ ID NO: 7 - DNA, 20 bp, Artificial Sequence
  Primer
ccgcctcccccatgccgccc

SEQ ID NO: 8 - DNA, 20 bp, Artificial Sequence
  Primer
cggggcggggcttcagctgc

SEQ ID NO: 9 - DNA, 20 bp, Artificial Sequence
  Primer
tcacttcgcttcccgcactg

SEQ ID NO: 10 - DNA, 20 bp, Artificial Sequence
  Primer
gccgtccatcttccttcata

SEQ ID NO: 11 - DNA, 20 bp, Artificial Sequence
  Primer
gctctcttgggtgcactgga

SEQ ID NO: 12 - DNA, 20 bp, Artificial Sequence
  Primer
caccgccttggcttgtcaca

(19) United States
(12) Patent Application Publication
(10) Pub. No.: US 2006/0264365 A1
(43) Pub. Date: Nov. 23, 2006

(54) Treatment of brain tissue damage

(76) Inventors: Hung Li, Taipei (TW); Woei-Cherng Shyu, Taipei (TW); Shinn-Zong Lin, Hualien (TW)

Correspondence Address: FISH & RICHARDSON PC, P.O. BOX 1022, MINNEAPOLIS, MN, 55440-1022, US

(21) Appl. No.: 11/131,896

(22) Filed: May 18, 2005

(51) Int. Cl. A61K 38/18

(52) U.S. Cl. 514/12

(57) ABSTRACT

Methods of treating brain tissue damage, increasing the expression level of a neuraltrophic factor in a cell, and enhancing angiogenesis in a tissue.

### BACKGROUND

Brain tissue damage, resulting either from injuries or disorders (e.g., neurodegenerative and cerebrovascular diseases), is a leading cause of long-term disability. Due to their pluripotency, embryonic stem cells (ES cells) hold a great promise for treating brain tissue damage (Lindvall et al., 2004, Nat Med., 10 Suppl: S42-50; and Taguchi et al., 2004, J. Clin. Invest.; 114(3):330-338). However, ethical and logistical considerations have hampered their use (Barinaga, 2000, Science, 287(5457):1421-1422; and Boer, 1994, J. Neurol., 242(1):1-13). Use of non-ES pluripotent cells has also been exploited. They include adult bone marrow mesenchymal stem cells or stromal cells (Sanchez-Ramos et al., 2000, Exp. Neurol., 164(2):247-256 and Woodbury et al., 2000, J. Neurosci. Res., 61(4):364-370) and umbilical cord blood cells (Galvin-Parton et al., 2003, Pediatr. Transplant. 2003; 7(2):83-85 and Ha et al., 2001 Neuroreport., 2(16):3523-3527). As requirements for in vitro expansion and HLA-matching have limited clinical applications of these cells, there is a need for an alternative method of treating brain tissue damage.

### SUMMARY

This invention is based, at least in part, on the discovery that brain tissue damage can be repaired by stromal cell-derived factor 1α (SDF-1α).

Accordingly, one aspect of the invention features a method of treating brain tissue damage (e.g., caused by an ischemic injury). The method includes administering, e.g., intracerebrally, to a subject in need thereof an effective amount of SDF-1α. SDF-1α protects cells in the brain tissue (e.g., neuronal cells or glial cells) from cell death by, e.g., repressing the activity of caspase-3 or increasing the expression level of a trophic factor. Examples of trophic factors include brain-derived neurotrophic factor (BDNF), glial-cell line derived neurotrophic factor (GDNF), and vascular endothelial growth factor (VEGF). SDF-1α also enhances migration of bone marrow-derived cells (e.g., hematopoietic stem cells) to the brain.

The invention also features a method of increasing the expression level of a trophic factor (e.g., GDNF, VEGF, or BDNF) in cells. The method includes contacting the cells with SDF-1α. Also within the scope of the invention is a method of enhancing angiogenesis in a tissue (e.g., the brain) of a subject. The method includes administering to a subject in need thereof an effective amount of SDF-1α.

“Treating” refers to administering a compound to a subject, who is suffering from or is at risk for developing brain tissue damage or a disorder causing such damage, with the purpose to cure, alleviate, relieve, remedy, prevent, or ameliorate the damage/disorder, the symptom of the damage/disorder, the disease state secondary to the damage/disorder, or the predisposition toward the damage/disorder. An “effective amount” refers to an amount of the compound that is capable of producing a medically desirable result as described above in a treated subject. The treatment method of this invention can be performed alone or in conjunction with other therapy.

The details of one or more embodiments of the invention are set forth in the description below. Other features, objects, and advantages of the invention will be apparent from the description and from the claims.

### DETAILED DESCRIPTION

It has been suggested that ES cells can be used to regenerate neuronal or glial cells in the brain and thereby treat brain tissue damage. However, their uses have been hampered by ethical and logistical restrictions. Due to fewer restrictions, peripheral blood hematopoietic stem cells (PBSCs) represent a promising alternative to other stem cells. Nonetheless, the number of PBSCs under a steady-state condition is very low. Also conventional stem cell transplantation requires surgical intervention and is associated with a high cell mortality rate.

One aspect of the present invention relates to treating brain tissue damage using SDF-1α. SDF-1α, a member of a chemokines family consisting of small secreted proteins (8-12kDa), is known to cause activation and migration of leukocytes (Baggiolini, 1998, Nature 392, 565-8; and Murdoch et al., 2000, Blood 95, 3032-43). SDF-1α receptor, CXCR4, is expressed in a wide variety of developmental neuronal tissues, including sympathetic ganglia, dorsal root sensory ganglion, midbrain, and granular cell layer of cerebellum (McGrath et al., 1999. Dev. Biol. 213, 442-56). In addition, there is evidence that expression of SDF-1α and CXCR4 is increased during neuropathogenesis induced by many forms of injury, including trauma, stroke and inflammation (Hill et al., 2004 J. Neuropathol. Exp. Neurol. 63, 84-96; Zheng et al., 1999, J. Neuroimmun. 98, 185-200; and Evert et al., 2001, J. Neurosci. 21, 5389-96).

As described in the examples below, SDF-1α unexpectedly enhanced targeting of autologous stem cells (e.g., PBSCs) to the injured brain; it protected neurons from cell death, induced neurotrophic factor expression, and promoted neurogenesis and angiogenesis in the brain. Thus, SDF-1α not only protects existing neurons in the brain, but also facilitates neural regeneration to replace damaged neurons.

Within the scope of this invention is a method of using SDF-1α to treat brain tissue damage. The method includes identifying a subject suffering from or being at risk for developing brain tissue damage. The subject can be a human or a non-human mammal, such as a cat, a dog, or a horse. Examples of the brain tissue damage includes those caused by a cerebral ischemia (e.g., stroke) or a neurodegenerative disease (e.g., Parkinson's disease, Alzheimer's disease, Spinocerebellar disease, or Huntington's disease). A subject to be treated can be identified by standard techniques for diagnosing the conditions or disorders of interest. The treatment method of this invention entails administering to the subject an effective amount of SDF-1α.

While many SDF-1α preparations can be used, highly purified SDF-1α is preferred. Examples of SDF-1α include mammalian SDF-1α (e.g., human SDF-1α) or SDF-1α having substantially the same biological activity as mammalian SDF-1α. All of naturally occurring SDF-1α, genetic engineered SDF-1α, and chemically synthesized SDF-1α can be used. SDF-1α obtained by recombinant DNA technology may be that having the same amino acid sequence as naturally occurring SDF-1α or an functionally equivalent thereof. A “functional equivalent” refers to a polypeptide derivative of a naturally occurring SDF-1α, e.g., a protein having one or more point mutations, insertions, deletions, truncations, a fusion protein, or a combination thereof. It posses at least one of the activities of SDF-1α, e.g., the ability to protect neurons from cell death, to induce neurotrophic factor expression, to target stem cells from bone marrow into peripheral blood, or to promote neurogenesis or angiogenesis in the brain. The term “SDF-1α” also covers chemically modified SDF-1α. Examples of chemically modified SDF-1α include SDF-1α subjected to conformational change, addition or deletion of a sugar chain, and SDF-1α to which a compound such as polyethylene glycol has been bound. Once purified and tested by standard methods, SDF-1α can be administered to a subject at, e.g., 1 to 100 μg/day/kg body weight once a day for 2-10 days, via any suitable routes.

The treatment method of this invention optionally includes administering to a subject an effective amount of PBSCs. Both heterologous and autologous PBSC can be used. In the former case, HLA-matching should be conducted to avoid or minimize host reactions. In the latter case, autologous PBSCs are enriched and purified from a subject to be treated before the cells are introduced back to the subject. In both cases, granulocyte-colony stimulating factor (G-CSF) can be used as the active ingredient to mobilize hematopoietic stem cells (HSCs) out of bone marrow so as to increase the number of stem cells in the peripheral blood, which home to the brain (HSCs, once in the peripheral blood, are called peripheral blood stem cells or PBSC). In a preferred embodiment, PBSCs are obtained from a subject as follows: A subject is first administered G-CSF to mobilize HSCs from bone marrow into the peripheral blood. After this enriching step, peripheral blood are collected and PBSCs purified.

To practice the treatment method of this invention, one can administer SDF-1α, as well as G-CSF, parenterally, inhalation spray, or via an implanted reservoir. The term “parenteral” as used herein includes intracerebral, subcutaneous, intracutaneous, intravenous, intramuscular, intraarterial, intraperitoneal, intrastemal, intrathecal, and intracranial injection or infusion techniques.

A sterile injectable composition (e.g., aqueous or oleaginous suspension) can be formulated according to techniques known in the art using suitable dispersing or wetting agents (such as, for example, Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or di-glycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents.

An inhalation composition can be prepared according to techniques well known in the art of pharmaceutical formulation and can be prepared as solutions in saline, employing benzyl alcohol, or other suitable preservatives, absorption promoters to enhance bioavailability, fluorocarbons, and/or other solubilizing or dispersing agents known in the art.

A topical composition can be formulated in form of oil, cream, lotion, ointment and the like. Suitable carriers for the composition include vegetable or mineral oils, white petrolatum (white soft paraffin), branched chain fats or oils, animal fats and high molecular weight alcohols (greater than C12). The preferred carriers are those in which the active ingredient is soluble. Emulsifiers, stabilizers, humectants and antioxidants may also be included as well as agents imparting color or fragrance, if desired. Additionally, transdermal penetration enhancers may be employed in these topical formulations. Examples of such enhancers can be found in U.S. Pat. Nos. 3,989,816 and 4,444,762. Creams are preferably formulated from a mixture of mineral oil, self-emulsifying beeswax and water in which mixture the active ingredient, dissolved in a small amount of an oil, such as almond oil, is admixed. An example of such a cream is one which includes about 40 parts water, about 20 parts beeswax, about 40 parts mineral oil and about 1 part almond oil. Ointments may be formulated by mixing a solution of the active ingredient in a vegetable oil, such as almond oil, with warm soft paraffin and allowing the mixture to cool. An example of such an ointment is one which includes about 30% almond and about 70% white soft paraffin by weight.

A carrier in a pharmaceutical composition must be “acceptable” in the sense of being compatible with the active ingredient of the formulation (and preferably, capable of stabilizing it) and not deleterious to the subject to be treated. For example, solubilizing agents, such as cyclodextrins (which form specific, more soluble complexes with one or more of active compounds of the extract), can be utilized as pharmaceutical excipients for delivery of the active compounds. Examples of other carriers include colloidal silicon dioxide, magnesium stearate, cellulose, sodium lauryl sulfate, and D&C Yellow # 10.

Suitable in vitro assays can be used to preliminarily evaluate the efficacy of an SDF-1α preparation in treating brain tissue damage. For example, one can measure the expression level of one of the trophic factors noted above. More specifically, a test preparation can be added to suitable cell cultures (e.g., primary cultures of rat or mouse cortical cells) and the expression level is determined. One then compares the level with a control level obtained in the absence of the preparation. If the level is higher than the control, the preparation is identified as being active for treating brain tissue damage. One can also evaluate the efficacy of an SDF-1α preparation by examining the preparation's effects on cell death according to standard methods. For example, one can measure the level of a protein involved in cell-death (e.g., caspase-3) or the activity of lactate dehydrogenase by the method described in Example 1 below. If the level or activity is lower than that obtained in the absence of the preparation, the preparation is determined to be active.

The preparation can further be examined for its efficacy in treating brain tissue damage by an in vivo assay. For example, the preparation can be administered to an animal (e.g., a mouse or rat model) having brain tissue damage or a disorder that causes brain tissue damage. The therapeutic effects of the preparation are then accessed according to standard methods (e.g., those described in Examples 3-7 below). To confirm efficacy in promoting cerebrovascular angiogenesis, one can examine the animal before and after the treatment by standard brain imaging techniques, such as computed tomography (CT), Doppler ultrasound imaging (DUI), magnetic resonance imaging (MRI), and proton magnetic resonance spectroscopy (1H-MRS).

One can also measure the expression level of a trophic factor or a cell death-related protein in a sample (e.g., cerebrospinal fluid) obtained from the animal before or after administering SDF-1α to confirm efficacy. The expression level can be determined at either the mRNA level or at the protein level. Methods of measuring mRNA levels in a tissue sample or a body fluid are well known in the art. To measure mRNA levels, cells can be lysed and the levels of mRNA in the lysates, whether purified or not, can be determined by, e.g., hybridization assays (using detectably labeled gene-specific DNA or RNA probes) and quantitative or semi-quantitative RT-PCR (using appropriate gene-specific primers). Alternatively, quantitative or semi-quantitative in situ hybridization assays can be carried out on tissue sections or unlysed cell suspensions using detectably (e.g., fluorescent or enzyme) labeled DNA or RNA probes. Additional mRNA-quantifying methods include the RNA protection assay (RPA) method and the serial analysis of gene expression (SAGE) method, as well as array-based technologies.

Methods of measuring protein levels in a tissue sample or a body fluid are also well known in the art. Some of them employ antibodies (e.g., monoclonal or polyclonal antibodies) that bind specifically to a target protein. In such assays, the antibody itself or a secondary antibody that binds to it can be detectably labeled. Alternatively, the antibody can be conjugated with biotin. Its presence can be determined by detectably labeled avidin (a polypeptide that binds to biotin). Combinations of these approaches (including “multi-layer sandwich” assays) can be used to enhance the sensitivity of the methodologies. Some protein-measuring assays (e.g., ELISA or Western blot) can be applied to body fluids or to lysates of cells, and others (e.g., immunohistological methods or fluorescence flow cytometry) can be applied to histological sections or unlysed cell suspensions. Appropriate labels include radionuclides (e.g., ¹²⁵I, ¹³¹I, ³⁵S, ³H, or ³²P), enzymes (e.g., alkaline phosphatase, horseradish peroxidase, luciferase, or β-glactosidase), fluorescent/luminescent agents (e.g., fluorescein, rhodamine, phycoerythrin, GFP, BFP, and Qdot™ nanoparticles supplied by the Quantum Dot Corporation, Palo Alto, Calif.). Other applicable methods include quantitative immunoprecipitation or complement fixation assays.

Based on the results from the assays described above, an appropriate dosage range and administration route can also be determined. The dosage required depends on the choice of the route of administration; the nature of the formulation; the nature of the patient's illness; the subject's size, weight, surface area, age, and sex; other drugs being administered; and the judgment of the attending physician. Suitable dosages are in the range of 0.001-100 mg/kg. Variations in the needed dosage are necessary in view of the variety of compounds available and the different efficiencies of various routes of administration. The variations can be adjusted using standard empirical routines for optimization as is well understood in the art. For example, a suitable intracerebral injection dosage is 1 to 100 μg/day/kg body weight; preferably 5-50 μg/day/kg body weight; and more preferably, 10-20 μg/day/kg body weight. Before or after administration, a subject can be examined to confirm treatment efficacy. To this end, one can use suitable standard tests or techniques described above, as well in the examples below.

The examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLE 1

Neuroprotective effect of SDF-1α was examined in primary cortical cultures. Primary cortical cells were prepared from the cerebral cortex of gestation day 17 embryos from Sprague-Dawley rats and seeded in 24-well plates as described in Murphy et al., 1990, FASEB J. 4, 1624-33. Four days later, the cultures were replenished with a minimum essential medium (MEM, GIBCO-BRL) containing 0.5 g/L BSA and N-2 supplement, 0.5×10⁻³ mol/L pyruvate, and antibiotics. On the seventh day, the culture medium was changed to a serum-free minimum essential medium containing 1×10⁻³ mol/L pyruvate, 1×10⁻³ mol/L glutamate, 0.5 g/L BSA, 0.3×10⁻³ mol/L KCl, and antibiotics.

The primary cortical neuron cultures were incubated with a medium containing 1 μg/mL SDF-1α (ProSpec-Tany TechnoGene, Israel) or a control medium for 20 minutes. Then, H₂O₂ (10⁻⁵ or 10⁻⁴ mol/L) was added to the cultures and incubated for 24 hours. The culture media were collected and subjected to lactate dehydrogenase (LDH) activity assays in the manner as described in Koh et al., J. Neurosci. Methods, 1987, 20, 83-90. Survived neurons were identified by MAP-2 immunostaining. More specifically, the primary cortical cell cultures were washed with PBS, fixed in 1% paraformaldehyde, immunostained by specific antibody against MAP-2 (1: 1000, Chemicon, Temecula, Calif.) and quantified according to the method described Wang et al., 2001, Stroke 32, 2170-8. The LDH activity and neuronal survival rate (represented by positive MAP-2 immunoreactivity) were obtained. It was found that treatment with SDF-1α (1 μg/mL) prior to H₂O₂ administration significantly reduced LDH activity in cultures exposed to 10⁻⁴ or 10⁻⁵ mol/L of H₂O₂. Also, SDF-1α (1 μg/mL) treatment also significantly prevent MAP-2 immunreactive cell loss due to H₂O₂.

### EXAMPLE 2

To determine the mechanism of SDF-1α's neuroprotection activity, the expression levels of a umber of trophic factors in the above-described primary cortical cultures were examined by RT-PCR. Primary cortical neuron cultures were treated with SDF-1α at different doses (0.01, 0.1, 1.0 and 10 μg/ml) and 10⁻⁵ mol/L H₂O₂ for 24 hours in the manner described above. Total RNA was extracted by an RNA-extraction kit (Qiagen, USA) according to the manufacturer's instructions. RT-PCR was conducted according to the method described in Shyu et al., 2004, Neurobiol. 24, 257-68). The specific PCR primers and the length of the amplified products are summarized in Table 1 below. GAPDH was used as an internal control.

TABLE 1
Sequence of PCR primers for neurotrophic factors

| | | |
|---|---|---|
| Factors | Sequence | PCR Fragment |

| | | |
|---|---|---|
| BDNF | sense-CAGTGGACATGTCCGGTGGGACGGTC | 533 bp |
| | anti-sense-TTCTTGGCAACGGCAACAAACCACAAC | |
| GDNF | sense-CCACACCGTTTAGCGGAATGC | 638 bp |
| | anti-sense-CGGGACTCTAAGATGAAGTTATGGG | |
| NGF | sense-GTTTTGGCCAGTGGTCGTGCAG | 498 bp |
| | anti-sense-CCGCTTGCTCCTGTGAGTCCTG | |
| TGF-β | sense-CCGCCTCCCCCATGCCGCCC | 710 bp |
| | anti-sense-CGGGGCGGGGCTTCAGCTGC | |
| FGF-II | sense-TCACTTCGCTTCCCGCACTG | 252 bp |
| | anti-sense-GCCGTCCATCTTCCTTCATA | |
| VEGF | sense-GCTCTCTTGGGTGCACTGGA | 431 bp |
| | anti-sense-CACCGCCTTGGCTTGTCACA | |

It was found that SDF-1α treatment significantly increased mRNA expression of GDNF, VEGF, and BDNF in a dose-dependent manner in comparison to the control. The ratio of BDNF, VEGF, and GDNF to GAPDH peaked at about a 2-fold increase in comparison to control. The up-regulation of GDNF and BDNF suggest that SDF-1α treatment protect brain tissues via the action of the neurotrophic factors. Also the increase in the expression of VEGF, an essential factor for angiogenesis, suggest that SDF-1α.promoted angiogenesis.

### EXAMPLE 3

Rats having cerebral ischemia were administered SDF-1α intracerebrally and examined for their neurological behavior.

More specifically, adult male Sprague-Dawley rats (weight>300 g) were anesthetized with chloral hydrate (0.4 g/kg, ip) and subjected to right middle cerebral artery (MCA) ligation and bilateral common carotid artery (CCAs) clamping in the manner described in Chen et al., 1986, Stroke 17, 738-43. Thirty minutes after MCA ligation, recombinant human SDF-1α (4 μg/4 μl PBS) (ProSpec-Tany TechnoGene, Israel) or vehicle (4 μl of PBS) were injected intracerebrally with through a 26-gauge Hamilton syringe (Hamilton Company, Reno, Nev.) into 3 cortical areas adjacent to the right MCA, 3.0 to 5.0 mm below the dura of each rat. The approximate coordinates for these three sites were (i) 1.0 to 2.0 mm anterior to the bregma and 2.5 to 3.0 mm lateral to the midline, (ii) 0.5 to 1.5 mm posterior to the bregma and 3.5 to 4.0 mm lateral to the midline, and (iii) 3.0 to 4.0 mm posterior to the bregma and 4.5 to 5.0 mm lateral to the midline. After 90 minutes, the 10-O suture on the MCA and arterial clips on CCAs were removed to allow reperfusion. Behavioral assessments were performed on SDF-1α treated (n=8) and control group rats (n=8) 3 days before cerebral ischemia, and 72 hours after cerebral ischemia. The tests measured (a) body asymmetry and (b) locomotor activity in the manner described in Chang, et al. Stroke 34, 558-64.

The results indicate that cerebral ischemia rats treated with SDF-1α exhibited significantly less body asymmetry than the control rats. The locomotor activity, such as vertical activity, vertical movement time, and number of vertical movements, significantly increased in rats receiving SDF-1α treatment compared with the control rats.

Systemic physiological parameters were analyzed in all rats in the manner described in Lin et al., 1999, Stroke 30, 126-33. It was found that intracerebral administration of SDF-1α did not alter systemic blood pressure, blood gases, blood glucose, or serum electrolyte levels. These data are summarized in Table 2.

TABLE 2
Effects of SDF-1α on physiological parameters

| Parameters | SDF-1α (n = 7) | Vehicle (n = 7) | p* |
|---|---|---|---|
| pH | 7.36 ± 0.013 | 7.351 ± 0.002 | 0.905 |
| PaCO₂, mm Hg | 46.12 ± 1.37 | 50.56 ± 2.7 | 0.251 |
| PaO₂, mm Hg | 90.11 ± 3.3 | 94.17 ± 3.0 | 0.215 |
| HCO₃⁻(10⁻³ mol/L) | 28.89 ± 1.37 | 25.07 ± 1.54 | 0.435 |
| Hematocrit, % | 45.02 ± 2.5 | 42.6 ± 3.9 | 0.291 |
| Hemoglobin (10 g/L) | 14.5 ± 0.48 | 15.99 ± 0.82 | 0.252 |
| Na⁺ (10⁻³ mol/L) | 139.1 ± 4.1 | 144.11 ± 2.12 | 0.667 |
| K⁺ (10⁻³ mol/L) | 4.06 ± 0.21 | 4.57 ± 0.36 | 0.810 |
| Ca⁺ (10⁻² g/L) | 4.01 ± 0.39 | 3.88 ± 1.03 | 0.565 |
| Glucose (10⁻² g/L) | 150.9 ± 29.2 | 142.91 ± 11.2 | 0.565 |
| MBP, mm Hg | 79.2 ± 8.41 | 80.1 ± 6.59 | 0.571 |
| HR, bpm | 397 ± 27 | 414 ± 17 | 0.610 |

MBP = mean blood pressure; HR = heart rate; *t test

The results suggest that the neuroprotective effect of SDF-1α did not result from changes in the physiological parameters listed above.

### EXAMPLE 4

The effects of SDF-1α on reducing infarction volumes was investigated in the cerebral ischemia rats described above. The rats were euthanized three days after cerebral ischemia and subjected to Triphenyltetrazolium chloride (TTC) staining. The rats were perfused intracardially with saline. The whole TTC staining procedure was described in Wang et al., 2001, Stroke 32, 2170-8. To minimize artifacts induced by post-ischemic edema in the infarcted tissue, the volume of infarction was calculated by a modified method based on that described by Lin. et al, 1993, Stroke 24, 117-21.

It was found that the SDF-1α-treated rats showed mild infarction. The average infarction volume of the eight SDF-1α-treated rats (71±15 mm³) was significantly smaller than that of the saline-treated control rats (174±17 mm³). The areas of the largest infarction for these two groups were 9.1±3.1 mm² (in treated rats) and 19±3.4 mm² (in controls), respectively. The infarcted slices per rat in the treated group (2.9±0.4 slices/rat) were also significantly fewer than those in the control group (6.1±0.3 slices/rat).

### EXAMPLE 5

Immuohistochemstry analysis was conducted on ischemic brain tissues from the above-descried rats to verify the neuroprotective effect of SDF-1α after cerebral ischemia. Specific antibodies that recognize neuron-specific proteins FITC-Neu-N (1:500, Chemicon, Temecula, Calif.) and MAP-2 (1:200, Chemicon, Temecula, Calif.) were used. It was found that, in the penumbric region surrounding the ischemic cores, the number of Neu-N and MAP-2 positive cells were significantly increased in SDF-1α-treated rats compared with the controls. In fact, ischemic brain tissue from the controls did not contain cells positive for MAP-2 or Neu-N in either the penumbric area or the ischemic core. These results indicate that SDF-1α protected neurons from cerebral ischemic damage.

To elucidated the neuroprotective mechanism, caspase-3 activity was examined. It is known that caspase-3 can be activated by cerebral ischemia. Twelve rats were subjected to MCA ligation and divided into two groups (six in each). The rats in the two groups respectively received SDF-1α and saline control in the manner described above. Eight hours after the MCA ligation, the rats were anesthetized by chloral hydrate and were perfused with 4% paraformaldehyde. Brains slices were prepared by a standard procedure and incubated with primary antibodies against caspase-3 (cleaved caspase-3 antibody, D175, dilution 1:100; Cell Signaling, Beverly, Mass.) and goat anti rabbit IgG conjugated with Cy3 (1:500, Jackson Immunoresearch, West Grove, Pa.) for 20 hours at 4° C., washed 3 times with PBS, and then observed with fluorescent microscopy (Axiovert 200M Carl Zeiss, Germany). The extent of apoptosis was represented as the number of caspase-3⁺ apoptotic cells per 10 High Power Field (HPFs). At least 20 fields were examined.

It was found that the penumbra surrounding the ischemic cores in SDF-1α treated rats contained few cells expressing activated caspase-3. Ischemic brain tissue from rats injected with the vehicle, however, contained many cells positive for activated caspase-3 in both the penumbra and the ischemic core. Quantitatively, rats treated with SDF-1α showed fewer cells positive for activated caspase-3 after ischemia than the control.

It is known that caspase-3 mediates cell death in cerebral ischemic models (Sasaki et al., 2000, Neurol. Res. 22, 223-8). The above results suggest that the neuroprotective mechanism of SDF-1α involved, at least in part, inhibition of the activation of caspase-3. More specifically, SDF-1α may exert its neuronal survival effect through CXCR4 signaling to induce a G inhibitory (Gi) protein-linked decrease in cAMP, which in turn downregulate Caspase-3 activation (Zheng et al., 1999, J Neuroimmunol 98, 185-200).

### EXAMPLE 6

To determine whether HSCs homed into the injured brain tissue of SDF-1α-treated rats, bromodeoxyuridine (BrdU) labeling was conducted to reveal HSCs, if any, in the brain according to the method described in Zhang et al., 2001, Neuroscience 105, 33-41. BrdU, a thymidine analog that is incorporated into the DNA of dividing cells during S-phase, was used for mitotic labeling (Sigma Chemical, St. Louis, Mo.). More specifically, the above-described rats were sacrificed three days after cerebral ischemia and subjected to BrdU staining with antibody against BrdU (1:400, Mannheim, Germany).

Cumulative BrdU labeling results revealed a few BrdU immunoreactive cells in the ipsilateral cortex near the infarcted boundary and subventricular region of ischemic hemisphere. BrdU immunoreactive cells were also found around the lumen of varying calibers of blood vessels in the perivascular portion of the ischemic hemisphere. In BrdU pulse labeling experiments, SDF-1α-treated rats (n=8) had significantly more BrdU immunoreactive cells than the control rats (n=8). These results suggest that SDF-1α stimulated stem cell to mobilize and home to brain.

### EXAMPLE 7

Double immunohistochemistry staining was performed on brain slices from each SDF-1α treated or control rat to determine whether the above-describe mobilized HSCs differentiated into neuronal, glial, or endothelial cells at ischemic sites in the brains. The staining was performed to examine the expression of glial fibrillary acidic protein (GFAP), von Willebrand factor (vWF), microtubule-associated protein 2 (MAP-2), and neuronal nuclei (Neu-N) according to the method described in Li et al., 2002, Neurology 59, 514-23. The antibodies used includes antibodies against BrdU (1:400, Mannheim, Germany) conjugated with FITC (1:500, Jackson Immunoresearch) or Cy3 (1:500, Jackson Immunoresearch), GFAP (1:400, Sigma) with Cy3 (1:500, Jackson Immunoresearch), MAP-2 (1:200, BM) with Cy3, Nestin (1:400, Sigma) with FITC, Neu-N (1:200, Chemicon) with FITC and vWF (1:400, Sigma) with Cy3. The tissue sections were examined under a Carl Zeiss LSM510 laser-scanning confocal microscope.

The result showed BrdU co-localized with Nestin, Neu-N, MAP-2, GFAP and vWF in some cells of the brains of the SDF-1α-treated rats. Ischemic cortical areas of SDF-1α treated-rats revealed more BrdU⁺ cells co-expressing Neu-N, Nestin, and MAP-2, as well as BrdU⁺/GFAP⁺ cells, than the saline-treated rats. Some BrdU⁺ cells showing vascular phenotypes (vWF⁺) were also found around the perivascular and endothelial regions of the ischemic hemispheres of SDF-1α-treated rats. These results suggest that SDF-1α enhanced neurogenesis and angiogenesis in vivo.

All of the above results support the role of SDF-1α/CXCR4 in adaptive early localized post-ischemic inflammation and later reorganization of the infarcted area. By attracting HSCs/PBSCs to the ischemic region, a SDF-1α/CXCR4 interaction may be directly involved in vascular remodeling, angiogenesis, and neurogenesis, thereby alleviating stroke symptoms. This chemotaxis may take place in a manner similar to the migration of leukocytes into damaged or inflamed tissues (Mariani et al., 2003, J. Immunol. Methods 273, 103-14). In addition, HSCs migrating to the ischemic hemisphere could create local chemical gradients and/or localized chemokine accumulation, dictating a directional response in endothelial, neuronal and glial progenitor cells (Yamaguchi et al, 2003, Circulation 107, 1322-8). In addition to inducing HSCs migration to ischemic regions, SDF-1α has also been shown to exert survival effects on cultured CD34⁺ cells and to regulate endothelial cell branching morphogenesis. Taken together, it is hypothesized that plasma levels of SDF-1α, released from damaged tissues, provides a host repair signal which in turn attracts mobilizing HSCs to repair the disordered tissue.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A method of treating brain tissue damage, comprising administering to a subject in need thereof an effective amount of stromal cell-derived factor 1α.

2. The method of claim 1, wherein the stromal cell-derived factor 1α protects a cell in the brain tissue from cell death.

3. The method of claim 2, wherein the stromal cell-derived factor 1α represses the activity of caspase-3 in the cell.

4. The method of claim 3, wherein the cell is a neuronal cell.

5. The method of claim 1, wherein the stromal cell-derived factor 1α enhances migration of a bone marrow-derived cell to the brain.

6. The method of claim 5, wherein the bone marrow-derived cell is a haematopoietic stem cell.

7. The method of claim 1, wherein the stromal cell-derived factor 1α increases the expression level of a trophic factor.

8. The method of claim 5, wherein the trophic factor is GDNF, VEGF, or BDNF.

9. The method of claim 1, wherein the stromal cell-derived factor 1α is administered intracerebrally.

10. The method of claim 1, wherein the brain tissue damage is caused by an ischemic injury.

11. The method of claim 10, wherein the stromal cell-derived factor 1α protects a cell in the brain tissue from cell death.

12. The method of claim 11, wherein the stromal cell-derived factor 1α represses the activity of caspase-3 in the cell.

13. The method of claim 12, wherein the cell is a neuronal cell.

14. The method of claim 10, wherein the stromal cell-derived factor 1α enhances migration of a bone marrow-derived cell to the brain.

15. The method of claim 14, wherein the bone marrow-derived cell is a haematopoietic stem cell.

16. The method of claim 10, wherein the stromal cell-derived factor 1α increases the expression level of a trophic factor.

17. The method of claim 16, wherein the trophic factor is GDNF, VEGF, or BDNF.

18. A method of increasing the expression level of a trophic factor in a cell, comprising contacting the cell with stromal cell-derived factor 1α.

19. The method of claim 18, wherein the trophic factor is GDNF, VEGF, or BDNF.

20. A method of enhancing angiogenesis in a tissue of a subject, which method comprises administering to a subject in need thereof an effective amount of stromal cell-derived factor 1α.

21. The method of claim 20, wherein the tissue is brain.
